(19) United States
(12) Patent Application Publication
(10) Pub. No.: US 2011/0282346 A1
(43) Pub. Date: Nov. 17, 2011

(54) Fracture Fixation Device, Tools and Methods

(75) Inventors: Trung Ho Pham, Santa Rosa, CA (US); Stephen R. McDaniel, Santa Rosa, CA (US); Charles L. Nelson, Santa Rosa, CA (US); Heber Saravia, Santa Rosa, CA (US)

(73) Assignee: Sonoma Orthopedic Products, Inc., Santa Rosa, CA (US)

(21) Appl. No.: 12/965,480

(22) Filed: Dec. 10, 2010

Related U.S. Application Data
(63) Continuation of application No. PCT/US09/46951, filed on Jun. 10, 2009.
(60) Provisional application No. 61/060,450, filed on Jun. 10, 2008.
(60) Provisional application No. 61/060,445, filed on Jun. 10, 2008.
(60) Provisional application No. 61/060,440, filed on Jun. 10, 2008.
(60) Provisional application No. 61/100,652, filed on Sep. 26, 2008.
(60) Provisional application No. 61/100,635, filed on Sep. 26, 2008.
(60) Provisional application No. 61/117,901, filed on Nov. 25, 2008.
(60) Provisional application No. 61/122,563, filed on Dec. 15, 2008.
(60) Provisional application No. 61/138,920, filed on Dec. 18, 2008.

(51) Int. Cl. A61B 17/58

(52) U.S. Cl. 606/62

(57) ABSTRACT

A bone fixation device is provided with an elongate body having a longitudinal axis and having a first state in which at least a portion of the body is flexible and a second state in which the body is generally rigid, an actuateable gripper disposed at a distal location on the elongated body, a hub located on a proximal end of the elongated body, and an actuator operably connected to the gripper to deploy the gripper from a retracted configuration to an expanded configuration. Methods of repairing a fracture of a bone are also disclosed. One such method comprises inserting a bone fixation device into an intramedullary space of the bone to place at least a portion of an elongate body of the fixation device in a flexible state on one side of the fracture and at least a portion of a hub on another side of the fracture, and operating an actuator to deploy at least one gripper of the fixation device to engage an inner surface of the intramedullary space to anchor the fixation device to the bone.

### CROSS-REFERENCE TO RELATED APPLICATIONS

This application is a Continuation-in-Part of U.S. application Ser. No. 11/383,269, titled “MINIMALLY INVASIVE ACTUABLE BONE FIXATION DEVICES”, filed May 15, 2006 which claims priority to U.S. Provisional Application No. 60/682,652, titled “METHOD AND SYSTEM FOR PROVIDING REINFORCEMENT OF BONES”, filed May 18, 2005. This application is also a Continuation-in-part of U.S. application Ser. No. 11/383,800 filed May 17, 2006, titled “DEPLOYABLE INTRAMEDULLARY STENT SYSTEM FOR REINFORCEMENT OF BONE” which claims priority to U.S. Provisional Application No. 60/682,652, titled “METHOD AND SYSTEM FOR PROVIDING REINFORCEMENT OF BONES”, filed May 18, 2005. This application is also a Continuation-in-Part of U.S. application Ser. No. 11/944,366, titled “FRACTURE FIXATION DEVICE, TOOLS AND METHODS”, filed Nov. 21, 2007 which claims priority to U.S. provisional applications: No. 60/867,011, titled “BONE REPAIR IMPLANT WITH CENTRAL RATCHETING GUIDEWIRE”, filed Nov. 22, 2006; No. 60/866,976, titled “SURGICAL TOOLS FOR USE TN DEPLOYING BONE REPAIR DEVICES,” filed Nov. 22, 2006; and No. 60/949,071, titled “FRACTURE FIXATION DEVICE, TOOLS AND METHODS”, filed Jul. 11, 2007.

This application claims priority of U.S. Provisional Application No. 61/060,440, titled “FRACTURE FIXATION DEVICE, TOOLS AND METHODS” filed Jun. 10, 2008; U.S. Provisional Application No. 61/060,445, titled “FRACTURE FIXATION DEVICE, TOOLS AND METHODS” filed Jun. 10, 2008; U.S. Provisional Application No. 61/060,450, titled “FRACTURE FIXATION DEVICE, TOOLS AND METHODS” filed Jun. 10, 2008; U.S. Provisional Application No. 61/100,635, titled “FRACTURE FIXATION DEVICE, TOOLS AND METHODS” filed Sep. 26, 2008; U.S. Provisional Application No. 61/100,652, titled “FRACTURE FIXATION DEVICE, TOOLS AND METHODS” filed Sep. 26, 2008; U.S. Provisional Application No. 61/122,563, titled “BONE FIXATION DEVICE, TOOLS AND METHODS” filed Dec. 15, 2008; U.S. Provisional Application No. 61/138,920, titled “BONE FIXATION DEVICE, TOOLS AND METHODS” filed Dec. 18, 2008; and U.S. Provisional Application No. 61/117,901, titled “BONE FRACTURE FIXATION SCREWS, SYSTEMS AND METHODS OF USE” filed Nov. 25, 2008.

### INCORPORATION BY REFERENCE

All publications and patent applications mentioned in this specification are herein incorporated by reference to the same extent as if each individual publication or patent application was specifically and individually indicated to be incorporated by reference

### BACKGROUND OF THE INVENTION

The present invention relates to devices, tools and methods for providing reinforcement of bones. More specifically, the present invention relates to devices, tools and methods for providing reconstruction and reinforcement of bones, including diseased, osteoporotic and fractured bones. The number and diversity sports and work related fractures are being driven by several sociological factors. The diversity of high energy sports has increased and the participation in these sports has followed the general trend of affluence and the resultant amount of time for leisure. High energy sports include skiing, motorcycle riding, snow mobile riding, snowboarding, mountain biking, road biking, kayaking, and all terrain vehicle (ATV) riding. As the general affluence of the economically developed countries has increased the number and age of people participating in these activities has increased. Lastly, the acceptance and ubiquitous application of passive restraint systems, airbags, in automobiles has created greater numbers of non-life threatening fractures. In the past, a person that might expire from a serious automobile accident now survives with multiple traumas and resultant fractures.

Bone fractures are a common medical condition both in the young and old segments of the population. However, with an increasingly aging population, osteoporosis has become more of a significant medical concern in part due to the risk of osteoporotic fractures. Osteoporosis and osteoarthritis are among the most common conditions to affect the musculoskeletal system, as well as frequent causes of locomotor pain and disability. Osteoporosis can occur in both human and animal subjects (e.g. horses). Osteoporosis (OP) and osteoarthritis (OA) occur in a substantial portion of the human population over the age of fifty. The National Osteoporosis Foundation estimates that as many as 44 million Americans are affected by osteoporosis and low bone mass, leading to fractures in more than 300,000 people over the age of 65. In 1997 the estimated cost for osteoporosis related fractures was $13 billion. That figure increased to $17 billion in 2002 and is projected to increase to $210-240 billion by 2040. Currently it is expected that one in two women, and one in four men, over the age of 50 will suffer an osteoporosis-related fracture. Osteoporosis is the most important underlying cause of fracture in the elderly. Also, sports and work-related accidents account for a significant number of bone fractures seen in emergency rooms among all age groups.

One current treatment of bone fractures includes surgically resetting the fractured bone. After the surgical procedure, the fractured area of the body (i.e., where the fractured bone is located) is often placed in an external cast for an extended period of time to ensure that the fractured bone heals properly. This can take several months for the bone to heal and for the patient to remove the cast before resuming normal activities.

In some instances, an intramedullary (IM) rod or nail is used to align and stabilize the fracture. In that instance, a metal rod is placed inside a canal of a bone and fixed in place, typically at both ends. See, for example, Fixion™ IM (Nail), www.disc-o-tech.com. Placement of conventional IM rods are typically a “line of sight” and require access collinear with the center line of the IM canal. Invariably, this line of sight access violates, disrupts, and causes damage to important soft tissue structures such as ligaments, tendons, cartilage, facia, and epidermis. This approach requires incision, access to the canal, and placement of the IM nail. The nail can be subsequently removed or left in place. A conventional IM nail procedure requires a similar, but possibly larger, opening to the space, a long metallic nail being placed across the fracture, and either subsequent removal, and or when the nail is not removed, a long term implant of the IM nail. The outer diameter of the IM nail must be selected for the minimum inside diameter of the space. Therefore, portions of the IM nail may not be in contact with the canal. Further, micro-motion between the bone and the IM nail may cause pain or necrosis of the bone. In still other cases, infection can occur. The IM nail may be removed after the fracture has healed. This requires a subsequent surgery with all of the complications and risks of a later intrusive procedure. In general, rigid IM rods or nails are difficult to insert, can damage the bone and require additional incisions for cross-screws to attach the rods or nails to the bone.

Some IM nails are inflatable. See, for example, Meta-Fix IM Nailing System, www.disc-o-tech.com. Such IM nails require inflating the rod with very high pressures, endangering the surrounding bone. Inflatable nails have many of the same drawbacks as the rigid IM nails described above.

External fixation is another technique employed to repair fractures. In this approach, a rod may traverse the fracture site outside of the epidermis. The rod is attached to the bone with trans-dermal screws. If external fixation is used, the patient will have multiple incisions, screws, and trans-dermal infection paths. Furthermore, the external fixation is cosmetically intrusive, bulky, and prone to painful inadvertent manipulation by environmental conditions such as, for example, bumping into objects and laying on the device.

Other concepts relating to bone repair are disclosed in, for example, U.S. Pat. No. 5,108,404 to Scholten for Surgical Protocol for Fixation of Bone Using Inflatable Device; U.S. Pat. No. 4,453,539 to Raftopoulos et al. for Expandable Intramedullary Nail for the Fixation of Bone Fractures; U.S. Pat. No. 4,854,312 to Raftopolous for Expanding Nail; U.S. Pat. No. 4,932,969 to Frey et al. for Joint Endoprosthesis; U.S. Pat. No. 5,571,189 to Kuslich for Expandable Fabric Implant for Stabilizing the Spinal Motion Segment; U.S. Pat. No. 4,522,200 to Stednitz for Adjustable Rod; U.S. Pat. No. 4,204,531 to Aginsky for Nail with Expanding Mechanism; U.S. Pat. No. 5,480,400 to Berger for Method and Device for Internal Fixation of Bone Fractures; U.S. Pat. No. 5,102,413 to Poddar for Inflatable Bone Fixation Device; U.S. Pat. No. 5,303,718 to Krajicek for Method and Device for the Osteosynthesis of Bones; U.S. Pat. No. 6,358,283 to Hogfors et al. for Implantable Device for Lengthening and Correcting Malpositions of Skeletal Bones; U.S. Pat. No. 6,127,597 to Beyar et al. for Systems for Percutaneous Bone and Spinal Stabilization, Fixation and Repair; U.S. Pat. No. 6,527,775 to Warburton for Interlocking Fixation Device for the Distal Radius; U.S. Patent Publication US2006/0084998 A1 to Levy et al. for Expandable Orthopedic Device; and PCT Publication WO 2005/112804 A1 to Myers Surgical Solutions, LLC et al. for Fracture Fixation and Site Stabilization System. Other fracture fixation devices, and tools for deploying fracture fixation devices, have been described in: US Patent Appl. Publ. No. 2006/0254950; U.S. Ser. No. 60/867,011 (filed Nov. 22, 2006); U.S. Ser. No. 60/866,976 (filed Nov. 22, 2006); and U.S. Ser. No. 60/866,920 (filed Nov. 22, 2006).

In view of the foregoing, it would be desirable to have a device, system and method for providing effective and minimally invasive bone reinforcement and fracture fixation to treat fractured or diseased bones, while improving the ease of insertion, eliminating cross-screw incisions and minimizing trauma.

### SUMMARY OF THE INVENTION

Aspects of the invention relate to embodiments of a bone fixation device and to methods for using such a device for repairing a bone fracture. The bone fixation device may include an elongate body with a longitudinal axis and having a flexible state and a rigid state. The device further may include a plurality of grippers disposed at longitudinally-spaced locations along the elongated body, a rigid hub connected to the elongated body, and an actuator that is operably-connected to the grippers to deploy the grippers from a first shape to an expanded second shape. The elongate body and the rigid hub may or may not be collinear or parallel.

In one embodiment, a bone fixation device is provided with an elongate body having a longitudinal axis and having a first state in which at least a portion of the body is flexible and a second state in which the body is generally rigid, an actuateable gripper disposed at a distal location on the elongated body, a hub located on a proximal end of the elongated body, and an actuator operably connected to the gripper to deploy the gripper from a retracted configuration to an expanded configuration.

Methods of repairing a fracture of a bone are also disclosed. One such method comprises inserting a bone fixation device into an intramedullary space of the bone to place at least a portion of an elongate body of the fixation device in a flexible state on one side of the fracture and at least a portion of a hub on another side of the fracture, and operating an actuator to deploy at least one gripper of the fixation device to engage an inner surface of the intramedullary space to anchor the fixation device to the bone.

According to aspects of the present disclosure, similar methods involve repairing a fracture of a metatarsal, metacarpal, sternum, tibia, rib, midshaft radius, ulna, olecranon (elbow), huberus, or distal fibula. Each of these bones have a distal and proximal segment, farthest and closest to the heart, respectively, and on opposite ends of a fracture. The method comprises creating an intramedullary channel, such that the channel traverses the fracture of the bone and comprises at least one segment that substantially follows a curved anatomical contour of the bone; and inserting a bone fixation device into the intramedullary channel and across the fracture of the bone, such that at least a portion of an elongate body of the fixation device in a flexible state is placed within the curved segment of the channel.

One embodiment of the present invention provides a low weight to volume mechanical support for fixation, reinforcement and reconstruction of one or other regions of the musculo-skeletal system in both humans and animals. The method of delivery of the device is another aspect of the invention. The method of delivery of the device in accordance with the various embodiments of the invention reduces the trauma created during surgery, decreasing the risks associated with infection and thereby decreasing the recuperation time of the patient. The framework may in one embodiment include an expandable and contractible structure to permit re-placement and removal of the reinforcement structure or framework.

In accordance with the various embodiments of the present invention, the mechanical supporting framework or device may be made from a variety of materials such as metal, composite, plastic or amorphous materials, which include, but are not limited to, steel, stainless steel, cobalt chromium plated steel, titanium, nickel titanium alloy (nitinol), super-elastic alloy, and polymethylmethacrylate (PMMA). The device may also include other polymeric materials that are biocompatible and provide mechanical strength, that include polymeric material with ability to carry and delivery therapeutic agents, that include bioabsorbable properties, as well as composite materials and composite materials of titanium and polyetheretherketone (PEEK™), composite materials of polymers and minerals, composite materials of polymers and glass fibers, composite materials of metal, polymer, and minerals.

Within the scope of the present invention, each of the aforementioned types of device may further be coated with proteins from synthetic or animal source, or include collagen coated structures, and radioactive or brachytherapy materials. Furthermore, the construction of the supporting framework or device may include radio-opaque markers or components that assist in their location during and after placement in the bone or other region of the musculo-skeletal systems.

Further, the reinforcement device may, in one embodiment, be osteo incorporating, such that the reinforcement device may be integrated into the bone.

In still another embodiment of the invention, a method of repairing a bone fracture is disclosed that comprises: accessing a fracture along a length of a bone through a bony protuberance at an access point at an end of a bone; advancing a bone fixation device into a space through the access point at the end of the bone; bending a portion of the bone fixation device along its length to traverse the fracture; and locking the bone fixation device into place within the space of the bone. The method can also include the step of advancing an obturator through the bony protuberance and across the fracture prior to advancing the bone fixation device into the space. In yet another embodiment of the method, the step of anchoring the bone fixation device within the space can be included.

An aspect of the invention discloses a removable bone fixation device that uses a single port of insertion and has a single-end of remote actuation wherein a bone fixation device stabilizes bone after it has traversed the fracture. The bone fixation device is adapted to provide a single end in one area or location where the device initiates interaction with bone. The device can be deployed such that the device interacts with bone. Single portal insertion and single-end remote actuation enables the surgeon to insert and deploy the device, deactivate and remove the device, reduce bone fractures, displace or compress the bone, and lock the device in place. In addition, the single-end actuation enables the device to grip bone, compresses the rigidizable flexible body, permits axial, torsional and angular adjustments to its position during surgery, and releases the device from the bone during its removal procedure. A removable extractor can be provided in some embodiments of the device to enable the device to be placed and extracted by deployment and remote actuation from a single end. The device of the invention can be adapted and configured to provide at least one rigidizable flexible body or sleeve. Further the body can be configured to be flexible in all angles and directions. The flexibility provided is in selective planes and angles in the Cartesian, polar, or cylindrical coordinate systems. Further, in some embodiments, the body is configured to have a remote actuation at a single end. Additionally, the body can be configured to have apertures, windings, etc. The device may be configured to function with non-flexible bodies for use in bones that have a substantially straight segment or curved segments with a constant radius of curvature. Another aspect of the invention includes a bone fixation device in that has mechanical geometry that interacts with bone by a change in the size of at least one dimension of a Cartesian, polar, or spherical coordinate system. Further, in some embodiments, bioabsorbable materials can be used in conjunction with the devices, for example by providing specific subcomponents of the device configured from bioabsorbable materials. A sleeve can be provided in some embodiments where the sleeve is removable, has deployment, remote actuation, and a single end. Where a sleeve is employed, the sleeve can be adapted to provide a deployable interdigitation process or to provide an aperture along its length through which the deployable interdigitation process is adapted to engage bone. In some embodiments, the deployable interdigitation process is further adapted to engage bone when actuated by the sleeve. In some embodiments, the bone fixation device further comprises a cantilever adapted to retain the deployable bone fixation device within the space. The sleeve can further be adapted to be expanded and collapsed within the space by a user. One end of the device can be configured to provide a blunt obturator surface adapted to advance into the bone. A guiding tip may also be provided that facilitates guiding the device through the bone. The device may be hollow and accept a guide wire. The guiding tip may facilitate placement of the device thereby providing a means to remove bone in its path (a helical end, a cutting end, or ablative end). The guiding tip may allow capture, interaction, or insertion into or around a tube on its internal or external surface. Further, the deployable bone fixation device can be adapted to receive external stimulation to provide therapy to the bone. The device can further be adapted to provide an integral stimulator which provides therapy to the bone. In still other embodiments, the device can be adapted to receive deliver therapeutic stimulation to the bone.

The devices disclosed herein may be employed in various regions of the body, including: spinal, cranial, thoracic, lower extremities and upper extremities. Additionally, the devices are suitable for a variety of breaks including, epiphyseal, metaphyseal, diaphyseal cortical bone, cancellous bone, and soft tissue such as ligament attachment and cartilage attachment.

The fracture fixation devices of various embodiments of the invention are adapted to be inserted through an opening of a fractured bone, such as the radius (e.g., through a bony protuberance on a distal or proximal end or through the midshaft) into an intramedullary canal of the bone. The device can be inserted in one embodiment in a line of sight manner collinear or nearly collinear, or parallel to the central axis of the intramedullary canal. In another embodiment the device can be inserted at an angle, radius, or tangency to the axis of the intramedullary canal. In another embodiment, the device can be inserted in a manner irrespective of the central axis of the intramedullary canal. In some embodiments, the fixation device has two main components, one configured component for being disposed on the side of the fracture closest to the opening and one component configured for being disposed on the other side of the fracture from the opening so that the fixation device traverses the fracture.

The device components cooperate to align, fix and/or reduce the fracture so as to promote healing. The device may be removed from the bone after insertion (e.g., after the fracture has healed or for other reasons), or it may be left in the bone for an extended period of time or permanently.

In some embodiments, the fracture fixation device has one or more actuatable bone engaging mechanisms such as anchors or grippers on its proximal and/or distal ends. These bone engaging mechanisms may be used to hold the fixation device to the bone while the bone heals. In another embodiment, the fracture fixation device has a plurality of actuatable bone engaging mechanisms such as grippers or anchors along its length. In another embodiment, the fracture fixation device has grippers or anchoring devices that interdigitate into the bone at an angle greater than zero degrees and less than 180 degrees to secure the bone segments of the fracture. In another embodiment the fracture fixation device has grippers or anchoring features that when activated from a state that facilitates insertion to a state that captures, aligns, and fixes the fracture, deploy in a geometry so that the resultant fixed bone is analogous or nearly identical, or identical to the geometry of the bone prior to the fracture, anatomical configuration. In one embodiment of the device, the flexible body allows insertion through tortuous paths within bone or created within bone. Upon activation from the state of insertion to the state of fixation, this device deforms so as to grip the bone upon multiple surfaces of the now collapsed, rigid, flexible body. In this collapsed state the device may be deform in such a way to re-achieve anatomical alignment of the bone. The device as described above can be fabricated so that it can have any cross sectional shape. Examples of cross sectional shapes include round, oval, square, rectangular, n-sided, where n is an integer from 1 to infinity, star shaped, spoke shaped,

In some embodiments, to aid in insertion into the intramedullary canal, at least one component of the fracture fixation device has a substantially flexible state and a substantially rigid state. Once in place, deployment of the device also causes the components to change from the flexible state to a rigid state to aid in proper fixation of the fracture. At least one of the components may be substantially rigid or semi-flexible. At least one component may provide a bone screw attachment site for the fixation device.

In some embodiments, to aid in insertion of the device into the intramedullary canal, the main component of the fracture fixation device has a substantially flexible state. Thereby, the device, prior to activation, may not have a rigid section. Once in place, deployment of the device also causes the components to change from the flexible state to a rigid state to aid in proper fixation of the fracture. At least one of the components may be semi-flexible. Placement of the device may be aided by a detachable rigid member such as a guide or outrigger. Placement of the device may be aided by removable rigid member such as a tube or guide wire. At least one component may provide a bone screw attachment site for the fixation device. At least one of the components of the device may allow a screw or compressive member to be attached along its axis to provide linear compression of one side of the fractured bone towards the other (e.g. compression of the distal segment towards the proximal segment or visa versa). At least one of the components of the device may accept a screw at an acute angle, and angle less than 30 degrees from the axis of the device that would allow compression of one side of the fractured bone towards the other. At least one of the components of the device may accept an alternately removable eyelet to accommodate a compressive device so as to compress one side of the fractured bone towards the other side.

Embodiments of the invention also provide deployment tools with a tool guide for precise alignment of one or more bone screws with the fracture fixation device. These embodiments also provide bone screw orientation flexibility so that the clinician can select an orientation for the bone screw(s) that will engage the fixation device as well as any desired bone fragments or other bone or tissue locations.

These and other features and advantages of the present invention will be understood upon consideration of the following detailed description of the invention and the accompanying drawings.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings of which:

FIG. 1 is a perspective view of an embodiment of a bone repair device implanted in a bone according to the invention.

FIG. 2 is another perspective view of the implanted device of FIG. 1.

FIG. 3 is a longitudinal cross-section view of the bone repair device of FIG. 1 in a non-deployed state.

FIG. 4 is a plan view of a combination deployment tool that may be used with the bone repair device of FIG. 1.

FIG. 5 is a cross-section view of the tool and device shown in FIG. 4.

FIG. 6 is a perspective view of the tool and device shown in FIG. 4.

FIG. 7 is a cross-section view of the implanted device of FIG. 1.

FIG. 8 is a perspective view of an alternative embodiment of the implanted device of FIG. 1.

FIG. 9 is a perspective view of another alternative embodiment of the implanted device of FIG. 1.

FIG. 10 is a perspective view of an exemplary rotary driver tool constructed according to aspects of the invention.

FIG. 11 is a proximally-looking exploded view showing the driver tool of FIG. 10.

FIG. 12 is a distally-looking exploded view showing the driver tool of FIG. 10.

FIG. 13 is a longitudinal cross-sectional view of the driver tool of FIG. 10.

FIG. 14 is a perspective view of the driver tool of FIG. 10 with the knob, cap and retaining ring removed to more clearly show the other components of the tool.

FIG. 15 is an exploded view showing a variation of the combination tool of FIG. 4.

FIG. 16 is a perspective view showing a variation of the bone repair device of FIG. 1.

FIG. 17A is a perspective view showing an alternative bone repair device.

FIG. 17B is a cross-section view showing the device of FIG. 17A.

FIG. 17C is an exploded view showing the device of FIG. 17A.

FIGS. 18A-18I depict x-rays which illustrate various steps of an exemplary surgical procedure.

FIGS. 19A-19B show an alternative embodiment fixation device in combination with a tension band.

### DETAILED DESCRIPTION OF THE INVENTION

By way of background and to provide context for the invention, it may be useful to understand that bone is often described as a specialized connective tissue that serves three major functions anatomically. First, bone provides a mechanical function by providing structure and muscular attachment for movement. Second, bone provides a metabolic function by providing a reserve for calcium and phosphate. Finally, bone provides a protective function by enclosing bone marrow and vital organs. Bones can be categorized as long bones (e.g. radius, femur, tibia and humerus) and flat bones (e.g. skull, scapula and mandible). Each bone type has a different embryological template. Further each bone type contains cortical and trabecular bone in varying proportions. The devices of this invention can be adapted for use in any of the bones of the body as will be appreciated by those skilled in the art.

Cortical bone (compact) forms the shaft, or diaphysis, of long bones and the outer shell of flat bones. The cortical bone provides the main mechanical and protective function. The trabecular bone (cancellous) is found at the end of the long bones, or the epiphysis, and inside the cortex of flat bones. The trabecular bone consists of a network of interconnecting trabecular plates and rods and is the major site of bone remodeling and resorption for mineral homeostasis. During development, the zone of growth between the epiphysis and diaphysis is the metaphysis. Finally, woven bone, which lacks the organized structure of cortical or cancellous bone, is the first bone laid down during fracture repair. Once a bone is fractured, the bone segments are positioned in proximity to each other in a manner that enables woven bone to be laid down on the surface of the fracture. This description of anatomy and physiology is provided in order to facilitate an understanding of the invention. Persons of skill in the art will also appreciate that the scope and nature of the invention is not limited by the anatomy discussion provided. Further, it will be appreciated there can be variations in anatomical characteristics of an individual patient, as a result of a variety of factors, which are not described herein. Further, it will be appreciated there can be variations in anatomical characteristics between bones which are not described herein.

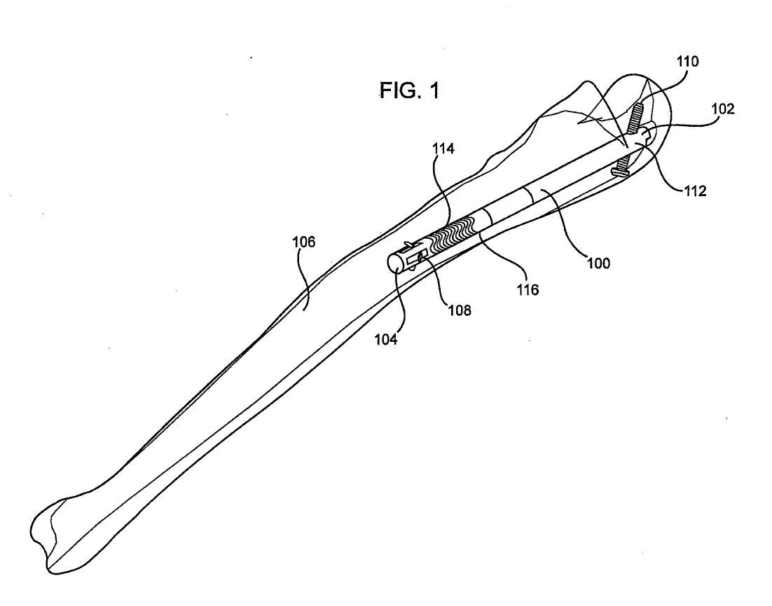

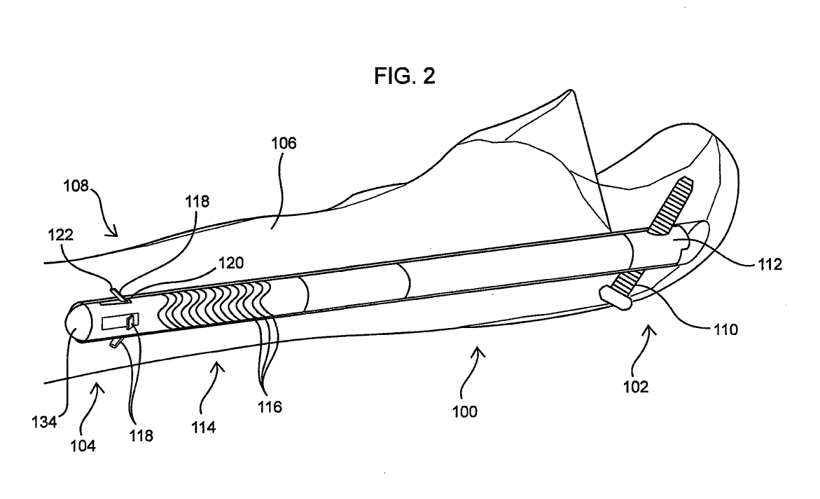

FIGS. 1 and 2 are perspective views of an embodiment of a bone repair device 100 having a proximal end 102 (nearest the surgeon) and a distal end 104 (further from surgeon) and positioned within the bone space of a patient according to the invention. In this example, device 100 is shown implanted in the upper (or proximal) end of an ulna 106. The proximal end and distal end, as used in this context, refers to the position of an end of the device relative to the remainder of the device or the opposing end as it appears in the drawing. The proximal end can be used to refer to the end manipulated by the user or physician. The distal end can be used to refer to the end of the device that is inserted and advanced within the bone and is furthest away from the physician. As will be appreciated by those skilled in the art, the use of proximal and distal could change in another context, e.g. the anatomical context in which proximal and distal use the patient as reference.

When implanted within a patient, the device can be held in place with suitable fasteners such as wire, screws, nails, bolts, nuts and/or washers. The device 100 is used for fixation of fractures of the proximal or distal end of long bones such as intracapsular, intertrochanteric, intercervical, supracondular, or condular fractures of the femur; for fusion of a joint; or for surgical procedures that involve cutting a bone. The devices 100 may be implanted or attached through the skin so that a pulling force (traction may be applied to the skeletal system).

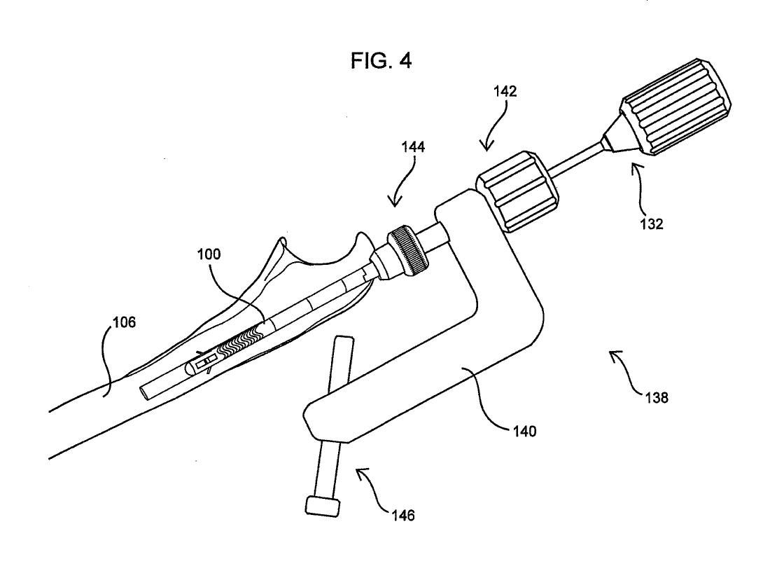

In the embodiment shown in FIG. 1, the design of the metaphyseal repair device 100 depicted is adapted to provide a bone engaging mechanism or gripper 108 adapted to engage target bone of a patient from the inside of the bone. As configured for this anatomical application, the device is designed to facilitate bone healing when placed in the intramedullary space within a post fractured bone. This device 100 has a gripper 108 positioned distally and shown deployed radially outward against the wall of the intramedullary cavity. On entry into the cavity, gripper 108 is flat and retracted (FIG. 4). Upon deployment, gripper 108 pivots radially outward and grips the diaphyseal bone from the inside of the bone. One or more screws 110 placed through apertures through the hub 112 lock the device 100 to the metaphyseal bone. Hence, the proximal end and or metaphysis and the distal end and or diaphysis are joined. The union between the proximal and distal ends may be achieved by the grippers 108 and 109 alone or in concert with screws 110 placed through hub 112. Hub 112 may be either at the distal or proximal end of the bone, in this case clavicle. A hub 112 may be at both ends of the device, there by allowing screws to be placed in the distal and proximal ends. A flexible-to-rigid body portion 114 may also be provided, and in this embodiment is positioned between grippers 108 and 109. The flexible-to-rigid body portion may be placed proximal or distal to both grippers, 108 and 109. It may be provided with cut 116 that is specific for the purpose and location of the device, as will be described in more detail below.

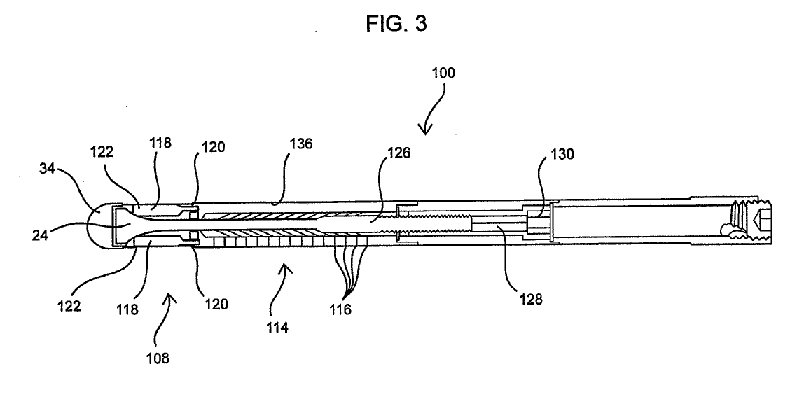

FIG. 3 shows a longitudinal cross-section of device 100 in a non-deployed configuration. In this embodiment, gripper 108 includes two pairs of opposing bendable members 118. Two of the bendable members 118 are shown in FIG. 3, while the other two (not shown in FIG. 3) are located at the same axial location but offset by 90 degrees. Each bendable member 118 has a thinned portion 120 that permits bending as the opposite distal end 122 of member 118 is urged radially outward, such that member 118 pivots about thinned portion 120. When extended, distal ends 122 of bendable members 118 contact the inside of the bone to anchor the distal portion of device 100 to the bone. In alternative embodiments (not shown), the gripper may comprise 1, 2, 3, 4, 5, 6 or more bendable members similar to members 118 shown. In some embodiments, gripper 109 may be made of a nickel-titanium alloy.

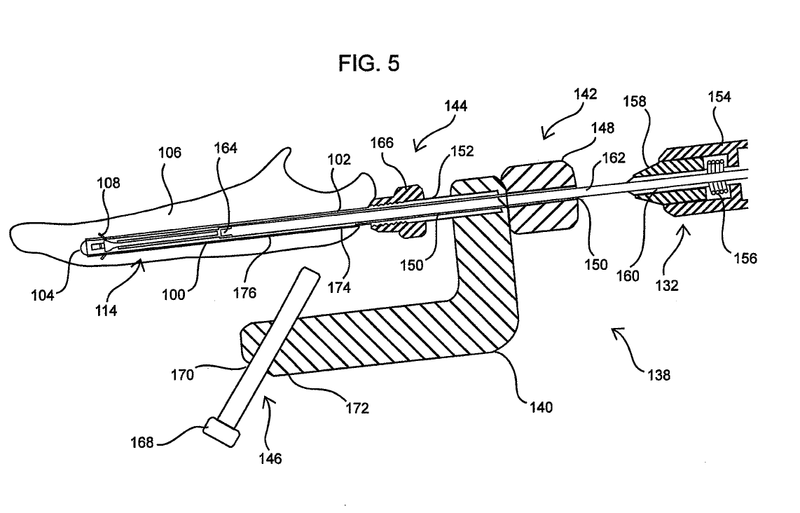

During actuation, bendable members 118 of gripper 108 are urged radially outward by a ramped surface on actuator head 124. Actuator head 124 is formed on the distal end of actuator 126. The proximal end of actuator 126 is threaded to engage a threaded bore of drive member 128. The proximal end of drive member 128 is provided with a keyed socket 130 for receiving the tip of a rotary driver tool 132 (shown in FIG. 5) through the proximal bore of device 100. As rotary driver tool 132 turns drive member 128, actuator 126 is drawn in a proximal direction to outwardly actuate gripper members 118. In an alternative embodiment, actuator 126 may be made of a super elastic alloy that when released from its insertion state it returns to its unstressed state thereby driving grippers 108 and 109 outward, shortening the device thereby compressing 518 into a rigid state.

Gripper 108 and the actuator head 124 may be reversed in their geometrical layout of the device. The gripper 108 may be drawn by the actuator 126 over the actuator head 124, thereby deflecting the bendable members, 118, outward. Similarly, the bendable members, 118, may be made of a super elastic or elastic or spring alloy of metal whereby the bendable members are predisposed in their set state in the insertion configuration, that being their smallest diameter. When the actuator head, 124, engages the super elastic, elastic or spring alloy of steel bendable members 118, a continuous force is imparted upon actuator head 124 such that the bendable members 118 return to their insertion geometry after the actuator head 124 is removed. Typical super elastic, elastic, or spring alloys of metals include spring steels and NiTi or nitinol. Conversely, bendable members 118 may be made of super elastic, elastic, or spring alloys of metal and set in their maximum outside diameter, in their deployed state. Actuator 124 and the rectangular apertures in 518 would work cooperatively to expose the bendable members 118. Since the bendable members 118 would be set in their maximum outside dimension and constrained within 518, upon exposure of 118 to the rectangular apertures, the bendable members would be driven by the material properties into the bone.

A hemispherical tip cover 134 may be provided at the distal end of the device as shown to act as a blunt obturator. This arrangement facilitates penetration of bone by device 100 while keeping the tip of device 100 from digging into bone during insertion.

As previously mentioned, device 100 may include one or more flexible-to-rigid body portions 114. This feature is flexible upon entry into bone and rigid upon application of compressive axial force provided by tensioning actuator 126. Various embodiments may be used, including dual helical springs whose inner and outer tubular components coil in opposite directions, a chain of ball bearings with flats or roughened surfaces, a chain of cylinders with flats, features, cones, spherical or pointed interdigitating surfaces, wavy-helical cut tubes, two helical cut tubes in opposite directions, linear wires with interdigitating coils, and bellows-like structures.

The design of the flexible-to-rigid tubular body portion 114 allows a single-piece design to maximize the transformation of the same body from a very flexible member that minimizes strength in bending to a rigid body that maximizes strength in bending and torque. The flexible member transforms to a rigid member when compressive forces are applied in the axial direction at each end, such as by an actuator. The body portion 114 is made, for example, by a near-helical cut 116 on a tubular member at an angle of incidence to the axis somewhere between 0 and 180 degrees from the longitudinal axis of the tubular body portion 114. The near-helical cut or wavy-helical cut may be formed by the superposition of a helical curve added to a cyclic curve that produces waves of frequencies equal or greater than zero per turn around the circumference and with cyclic amplitude greater than zero. The waves of one segment nest with those on either side of it, thus increasing the torque, bending strength and stiffness of the tubular body when subjective to compressive forces. The tapered surfaces formed by the incident angle allow each turn to overlap with the segment on either side of it, thus increasing the bending strength when the body is in compression. Additionally, the cuts can be altered in depth and distance between the cuts on the longitudinal axis along the length of body portion 114 to variably alter the flexible-to-rigid characteristics of the tubular body along its length.

The cuts 116 in body portion 114 allow an otherwise rigid member to increase its flexibility to a large degree during deployment. The tubular member can have constant or varying internal and external diameters. This design reduces the number of parts of the flexible-to-rigid body portion of the device and allows insertion and extraction of the device through a curved entry port in the bone while maximizing its rigidity once inserted. Application and removal of compressive forces provided by a parallel member such as wire(s), tension ribbons, a sheath, or actuator 126 as shown will transform the body from flexible to rigid and vice versa.

The flexible to rigid bodies may have a polygonal cross sectional geometry having any suitable number of sides from 1 to infinity. The flexible-to-rigid body may be cut in a specific way so that upon activation it conforms to a specific shape. The resultant shape may resemble or match the original anatomical shape of the bone. The resultant shape may provide specific translational actions so as to improve the healing of bone or create a resultant bone-implant construct that promotes a desired resultant geometry or effect. These resultant geometries may be bone lengthening where growth of the bone is improper, bone rotation to remediate poor pronation, supination, deflection, extension, deviation, or inclination of an appendage or joint. The shape of the flexible-to-rigid body may be devised or designed from x-ray or CT scans of the contralateral unaffected anatomy to return the affected anatomy to its original anatomical configuration or match the existing contralateral configuration.

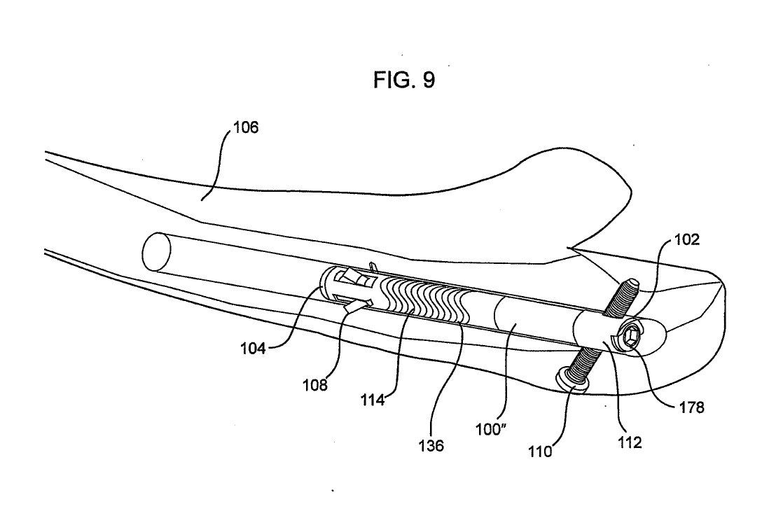

In operation, as actuator 126 is tightened, gripper members 118 are extended radially outwardly. Once the distal ends of gripper members 118 contact bone and stop moving outward, continued rotation of actuator 126 draws the proximal end 102 and the distal end 104 of device 100 closer together until cuts 116 are substantially closed. As this happens, body portion 114 changes from being flexible to rigid to better secure the bone fracture(s), as will be further described below. Rotating actuator 126 in the opposite direction causes body portion 114 to change from a rigid to a flexible state, such as for removing device 100 if needed in the initial procedure or during a subsequent procedure after the bone fracture(s) have partially or completely healed. Body portion 114 may be provided with a solid longitudinal portion 136 (as best seen in FIGS. 3 and 9) such that cuts 116 are a series of individual cuts each traversing less than 360 degrees in circumference, rather than a single, continuous helical cut. This solid portion 136 can aid in removal of device 100 by keeping body portion 114 from undesirably extending like a spring.

FIG. 4 illustrates a combination tool 138 useful for inserting device 100, actuating gripper 108, compressing flexible-to-rigid body portion 114, approximating the fracture in bone 106, aligning anchor screw(s) 110, and removing device 100, if desired. In this exemplary embodiment, tool 138 includes an L-shaped body 140 that mounts the other components of the tool and also serves as a handle. The main components of tool 138 are a device attachment portion 142, a rotary driver 132, an approximating driver 144, and a screw alignment portion 146.

FIG. 5 shows a cross-section of the tool 138 and device 100 illustrated in FIG. 4. As shown, device attachment portion 142 includes a knob 148 rigidly coupled to a tube 150 which is rotatably mounted within sleeve 152. Sleeve 152 in turn is fixedly mounted to tool body 140. The distal end of tube 150 is provided with external threads for engaging the internal threads on the proximal end of device 100. As best seen in FIG. 4, both the distal end of sleeve 152 and the proximal end of device 100 may be provided with steps that inter-engage to prevent device 100 from rotating with respect to sleeve 152. The step(s) can be semicircular or of any other suitable geometrical configuration so that the insertion tool and hub are keyed relative to each other for alignment and secure positioning. With this arrangement, device 100 can be prevented from rotating when it is secured to tool 138 by tube 150 of device attachment portion 142. The mating semicircular steps also serve to position device 100 in a particular angular orientation with respect to tool 138 for aligning screws with screw holes, as will be later described.

Rotary driver 132 may be used to actuate gripper 108 and compress flexible-to-rigid body portion 114 after device 100 is inserted into bone 106. Driver 132 may also be used to allow body portion 114 to decompress and gripper 108 to retract if removal of device 100 from bone 106 is desired. In the embodiment shown, driver 132 includes knob 154, spring 156, hub 158, bushing 160 and shaft 162. The distal end of shaft 162 is provided with a mating tip 164, such as one having a hex-key shape, for engaging with keyed socket 130 of device 100 (best seen in FIG. 3), such that turning driver shaft 162 turns actuator 126, as described above.

The proximal end of shaft 162 may be fitted with a bushing 160, such as with a press-fit. Hub 158 may be secured over bushing 160, such as with a pin through bushing 160 and shaft 162. In this embodiment, knob 154 is rotatably mounted over hub 158 and bushing 160 such that knob 154 can rotate independently from shaft 162. A torsion spring 156 may be used to couple knob 154 to hub 158 as shown to create a torque limiting and/or torque measuring driver. With this indirect coupling arrangement, as knob 154 is rotated about shaft 162, spring 156 urges hub 158 and shaft 162 to rotate in the same direction. Rotational resistance applied by device 100 to shaft tip 164 will increase in this embodiment as gripper 108 engages bone 106, and flexible-to-rigid body portion 114 compresses. As more torque is applied to knob 154, it will advance rotationally with respect to hub 158 as torsion spring 156 undergoes more stress. Markings may be provided on knob 154 and hub 158 to indicate the torque being applied. In this manner, a surgeon can use driver 132 to apply torque to device 100 in a predetermined range. This can help ensure that gripper 108 is adequately set in bone 106, body portion 114 is sufficiently compressed, and excessive torque is not being applied that might damage device 100, bone 106 or cause slippage therebetween. A slip clutch or other mechanism may be provided to allow the applied torque to be limited or indicated. For example, driver 132 may be configured to “click” into or out of a detent position when a desired torque is reached, thus allowing the surgeon to apply a desired torque without needing to observe any indicia on the driver. In alternative embodiments, the driver knob may be selectably or permanently coupled to shaft 162 directly.

After device 100 is inserted in bone 106 and deployed with tool 138 as described above, the approximating driver portion 144 of tool 138 may be used to compress one or more fractures in bone 106. Approximating driver 144 includes knob 166 located on sleeve 152. Knob 166 may be knurled on an outer circumference, and have threads on at least a portion of its axial bore. The internal threads of knob 166 engage with mating external threads on sleeve 152 such that when knob 152 is rotated it advances axially with respect to sleeve 152. When device 100 is anchored in bone 106, sleeve 152 is prevented from moving away from the bone. Accordingly, as knob 152 is advanced axially toward bone 106, it serves to approximate bone fractures located between gripper 108 and knob 152. Suitable thread pitch and knob circumference may be selected to allow a surgeon to supply a desired approximating force to bone 106 by using a reasonable rotation force on knob 152. In alternative embodiments (not shown), a torque indicating and/or torque limiting mechanism as described above may be incorporated into approximating driver 144.

As previously indicated, tool 138 may also include a screw alignment portion 146. In the embodiment depicted in the figures, alignment portion 146 includes a removable alignment tube 168 and two bores or apertures 170 and 172 through tool body 140. In alternative embodiments (not shown), a single bore or more than two bores may be used, with or without the use of separate alignment tube(s).

In operation, alignment tube 168 is first received in bore 170 as shown. In this position, tube 168 is in axial alignment with angled hole 174 at the distal end 102 of device 100. As described above, the mating semicircular steps of device 100 and sleeve 152 position angled hole 174 in its desired orientation. With this arrangement, a drill bit, screw driver, screw and/or other fastening device or tool may be inserted through the bore of tube 168 such that the device(s) are properly aligned with hole 174. The outward end of alignment tube 168 may also serve as a depth guide to stop a drill bit, screw and/or other fastener from penetrating bone 106 beyond a predetermined depth. Hole 174 may be tapped to interfere with the bone screw, interlocking pin, or transverse bone attachment member so that there is mechanical interference between the hub 112 and the bone screw, etc. This provides a means of capturing the screw so that, over time, it does not back out or translate away or into the hub unexpectedly.

Alignment tube 168 may be withdrawn from bore 170 as shown, and inserted in bore 172. In this position, tube 168 aligns with hole 176 of device 100. As described above, a drill bit, screw driver, screw and/or other fastening device may be inserted through the bore of tube 168 such that the device(s) are properly aligned with hole 176.

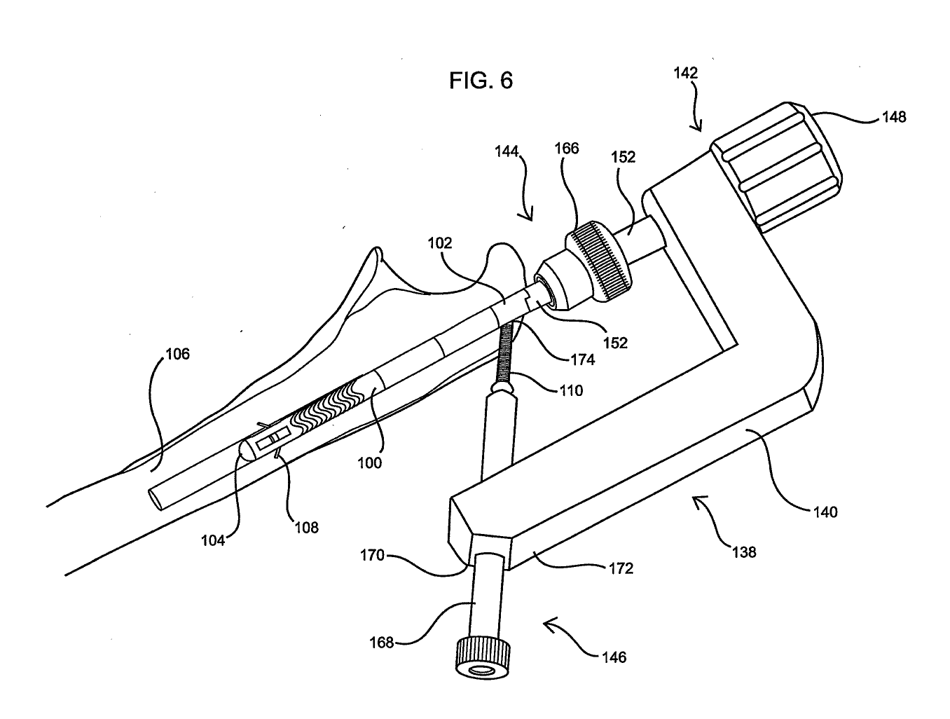

FIG. 6 shows alignment tube 168 of tool 138 aligning screw 110 with angled hole 174 at the distal end of device 110, as described above.

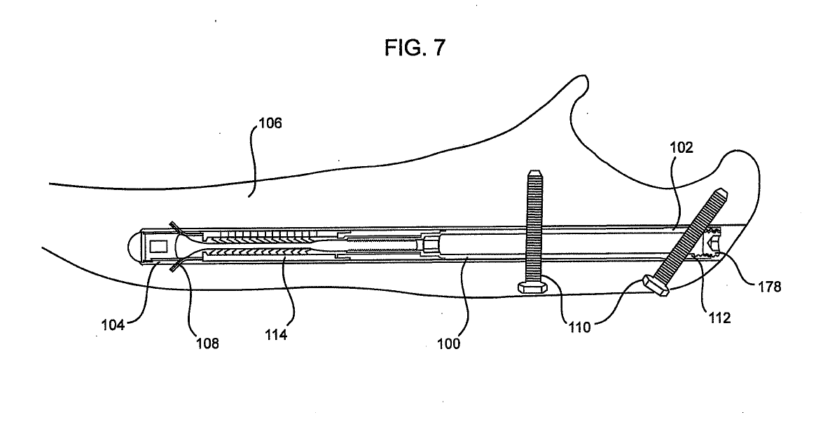

FIG. 7 shows a first screw 110 received through angled hole 174 and a second screw 110 received through hole 176 in device 100 and into bone 106. Screws 110 may be installed manually or with the aid of tool 138 as described above. The heads of screws 110 may be configured to be self-countersinking such that they remain substantially beneath the outer surface of the bone when installed, as shown, so as to not interfere with adjacent tissue. In this embodiment, the proximal end 102 of device 100 is secured to bone 106 with two screws 110, and the distal end 104 is secured by gripper 108. In this manner, any bone fractures located between the proximal screw 110 and distal gripper 108 may be approximated and rigidly held together by device 100. In alternative embodiments (not shown), more than one gripper may be used, or only screws or other fasteners without grippers may be used to secure device 100 within bone 106. For example, the device shown in FIG. 1 could be configured with a second gripper located between screw 110 and the middle of the device if the fracture is located more at the mid-shaft of the bone. Similarly, more than two screws or other fasteners may be used, or only grippers without fasteners may be used. In various embodiments, holes such as 174 and 176 as shown and described above can be preformed in the implantable device. In other embodiments, some or all of the holes can be drilled or otherwise formed in situ after the device is implanted in the bone.

Once device 100 is secured within bone 106, combination tool 138 may be removed by turning knob 148 to disengage threads of tube 150 from threads within the proximal end 102 of device 100. An end plug 178 may be threaded into the proximal end 102 of device 100 to prevent ingrowth of tissue into implanted device 100. Device 100 may be left in bone 106 permanently, or it may be removed by performing the above described steps in reverse. In particular, plug 178 is removed, tool 138 is attached, screws 110 are removed, gripper 108 is retracted, and device 100 is pulled out using tool 138.

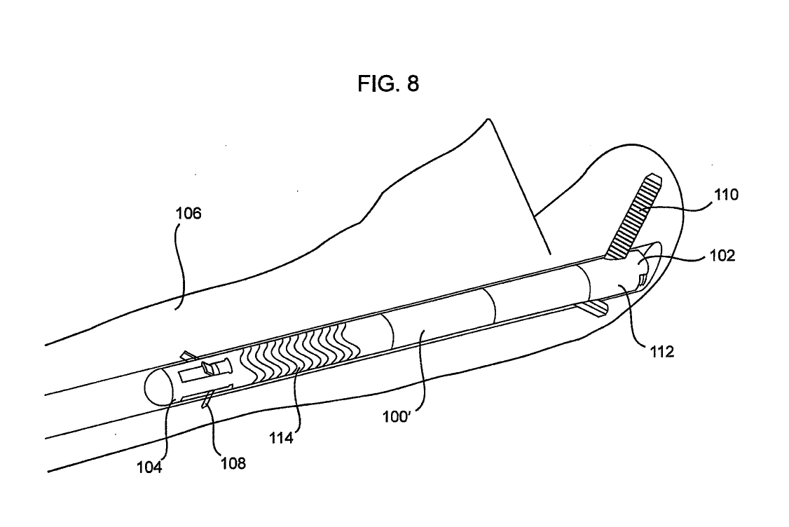

FIGS. 8 and 9 show alternative embodiments similar to device 100 described above. Device 100′ shown in FIG. 8 is essentially identical to device 100 described above but is shorter in length and utilizes a single anchor screw 110 at its proximal end 102. Device 100″ shown in FIG. 9 is similar to device 100′, but is shorter still. In various embodiments, the devices may be configured to have a nominal diameter of 4 mm, 5 mm or 6 mm. It is envisioned that all three devices 100, 100′ and 100″ may each be provided in all three diameters such that the chosen device is best suited for the particular fracture(s) and anatomy in which it is implanted.

In accordance with the various embodiments of the present invention, the device may be made from a variety of materials such as metal, composite, plastic or amorphous materials, which include, but are not limited to, steel, stainless steel, cobalt chromium plated steel, titanium, nickel titanium alloy (nitinol), superelastic alloy, and polymethylmethacrylate (PMMA). The device may also include other polymeric materials that are biocompatible and provide mechanical strength, that include polymeric material with ability to carry and delivery therapeutic agents, that include bioabsorbable properties, as well as composite materials and composite materials of titanium and polyetheretherketone (PEEK™), composite materials of polymers and minerals, composite materials of polymers and glass fibers, composite materials of metal, polymer, and minerals.

Within the scope of the present invention, each of the aforementioned types of device may further be coated with proteins from synthetic or animal source, or include collagen coated structures, and radioactive or brachytherapy materials. Furthermore, the construction of the supporting framework or device may include radio-opaque markers or components that assist in their location during and after placement in the bone or other region of the musculo-skeletal systems.

Further, the reinforcement device may, in one embodiment, be osteo incorporating, such that the reinforcement device may be integrated into the bone.

In a further embodiment, there is provided a low weight to volume device deployed in conjunction with other suitable materials to form a composite structure in-situ. Examples of such suitable materials may include, but are not limited to, bone cement, high density polyethylene, Kapton™, polyetheretherketone (PEEK™), and other engineering polymers.

Once deployed, the device may be electrically, thermally, or mechanically passive or active at the deployed site within the body. Thus, for example, where the device includes nitinol, the shape of the device may be dynamically modified using thermal, electrical or mechanical manipulation. For example, the nitinol device may be expanded or contracted once deployed, to move the bone or other region of the musculo-skeletal system or area of the anatomy by using one or more of thermal, electrical or mechanical approaches.

It is contemplated that the inventive implantable device, tools and methods may be used in many locations within the body. Where the proximal end of a device in the anatomical context is the end closest to the body midline and the distal end in the anatomical context is the end further from the body midline, for example, on the humerus, at the head of the humerus (located proximal, or nearest the midline of the body) or at the lateral or medial epicondyle (located distal, or furthest away from the midline); on the radius, at the head of the radius (proximal) or the radial styloid process (distal); on the ulna, at the head of the ulna (proximal) or the ulnar styloid process (distal); for the femur, at the greater trochanter (proximal) or the lateral epicondyle or medial epicondyle (distal); for the tibia, at the medial condyle (proximal) or the medial malleolus (distal); for the fibula, at the neck of the fibula (proximal) or the lateral malleoulus (distal); the ribs; the clavicle; the phalanges; the bones of the metacarpus; the bones of the carpus; the bones of the metatarsus; the bones of the tarsus; the sternum and other bones, the device may be adapted and configured with adequate internal dimension to accommodate mechanical fixation of the target bone and to fit within the anatomical constraints. As will be appreciated by those skilled in the art, access locations other than the ones described herein may also be suitable depending upon the location and nature of the fracture and the repair to be achieved. Additionally, the devices taught herein are not limited to use on the long bones listed above, but can also be used in other areas of the body as well, without departing from the scope of the invention. It is within the scope of the invention to adapt the device for use in flat bones as well as long bones.

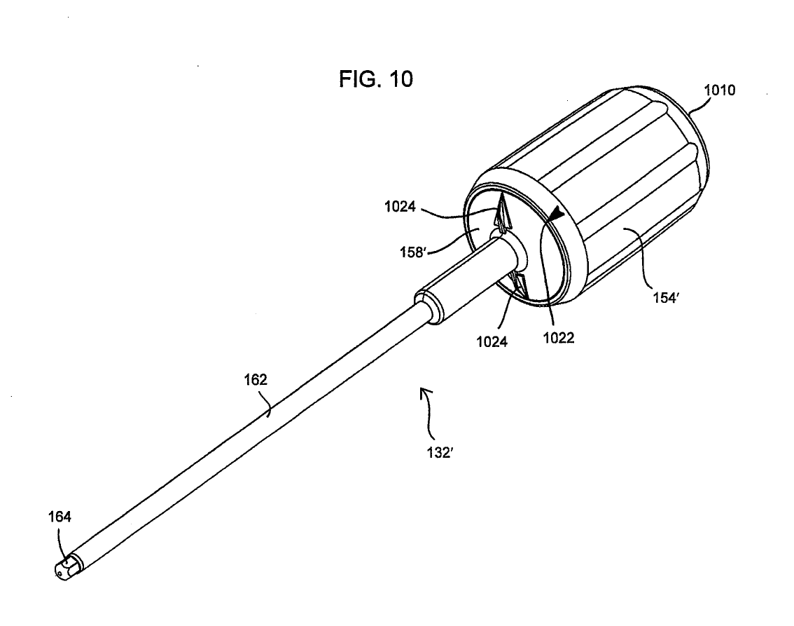

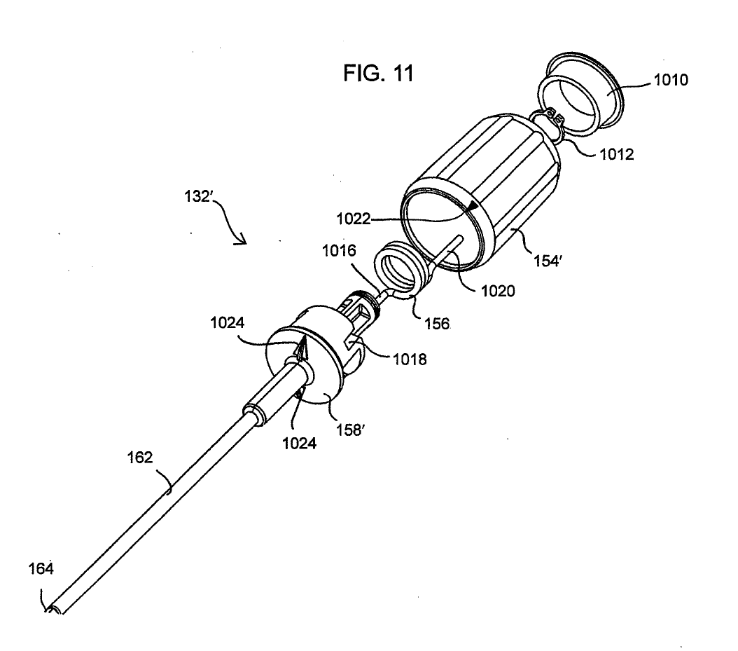

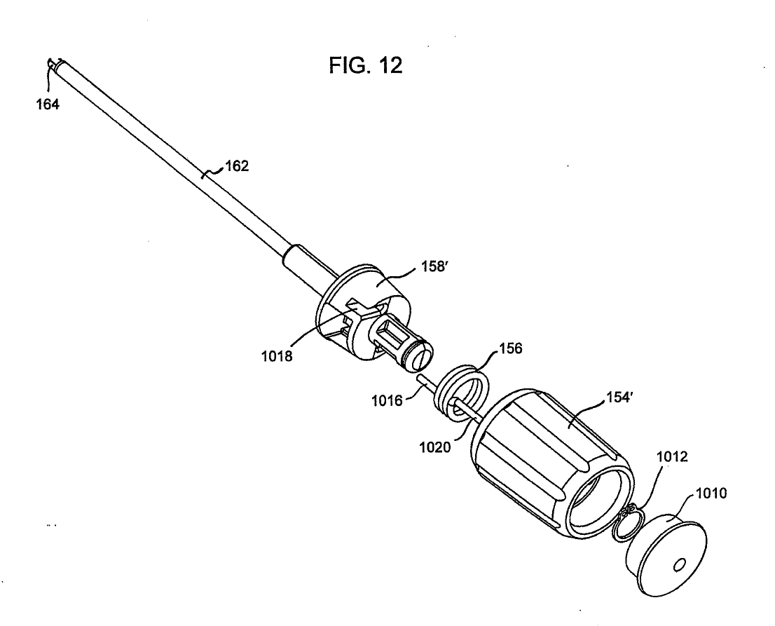

FIGS. 10-14 show further details of another exemplary rotary driver tool 132′, similar to the driver tool 132 shown in FIG. 5, constructed according to aspects of the invention. Driver tool 132′ may be used to actuate gripper 108 and compress flexible-to-rigid body portion 114 (both shown in FIG. 5) after device 100 is inserted into bone 106. Driver 132′ may also be used to allow body portion 114 to decompress and gripper 108 to retract if removal of device 100 from bone 106 is desired. In the embodiment shown in FIGS. 10-14, driver 132′ includes cap 1010, retaining ring 1012, knob 154′, spring 156, hub 158′, and shaft 162. The distal end of shaft 162 is provided with a mating tip 164, such as one having an Allen, Torx®, Philips, or similar shape, for engaging with keyed socket 130 of device 100 (best seen in FIG. 3), such that turning driver shaft 162 turns actuator 126, as previously described.

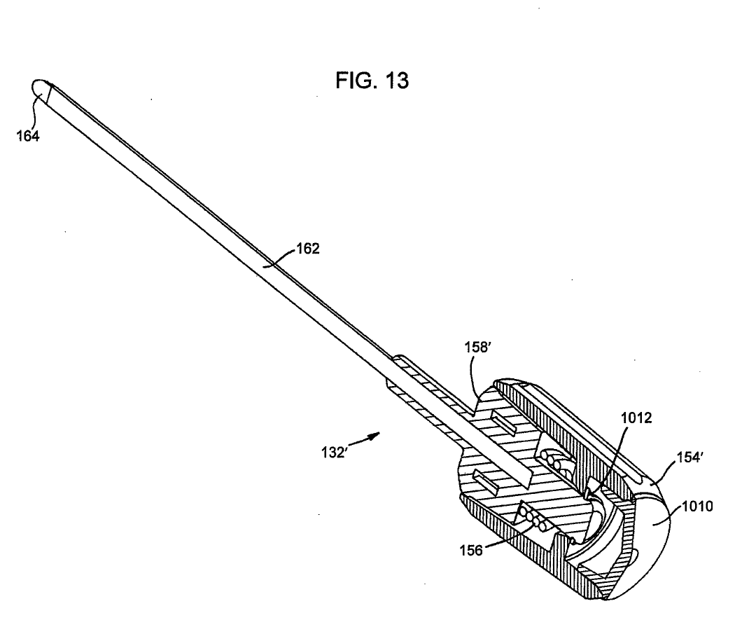

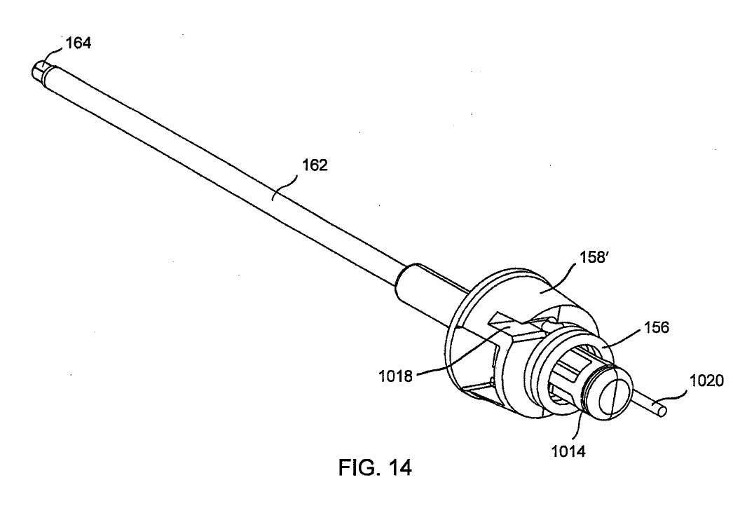

The proximal end of shaft 162 may be integrally formed with hub 158′, such as with an insert mold process. In this embodiment, knob 154′ is rotatably mounted over hub 158′ such that knob 154′ can rotate independently from hub 158′ and shaft 162. Knob 154′ may be restrained from axial movement in the proximal direction (i.e. away from shaft 162) by retaining ring 1012. In this embodiment, retaining ring 1012 engages with groove 1014 in the proximal end of hub 158′, shown in FIG. 14. A torsion spring 156 may be used to couple knob 154′ to hub 158′ as shown. More specifically, distal leg 1016 of spring 156 engages with slot 1018 in hub 158′, and proximal leg 1020 engages with a similar feature (not shown) within knob 154′.

With the indirect coupling arrangement just described, as knob 154′ is rotated about hub 158′ and shaft 162, spring 156 urges hub 158′ and shaft 162 to rotate in the same direction. Rotational resistance applied by device 100 to shaft tip 164 will increase in this embodiment as gripper 108 engages bone 106, and flexible-to-rigid body portion 114 compresses (see FIG. 5). As more torque is applied to knob 154′, it will advance rotationally with respect to hub 158′ as torsion spring 156 undergoes more stress.

A pair of marks 1022 may be provided on knob 154′ for aligning with a corresponding pair of marks 1024 on hub 158′ when a predetermined torque is applied to knob 154′. In this manner, a surgeon can use driver 132′ to apply an exact amount of torque to device 100. This can help ensure that gripper 108 is adequately set in bone 106, body portion 114 is sufficiently compressed, and excessive torque is not being applied that might damage device 100, bone 106 or cause slippage therebetween.

Driver 132′ may be calibrated by not applying marks 1022 to knob 154′ until after the driver is fabricated, assembled and calibrated. Marks 1024 may be molded onto the distal surface of hub 158′ as shown during fabrication. After tool 132′ is assembled, either tool tip 164 or knob 154′ can be held in a stationary position while a predetermined torque is applied to the other component, such as with a precisely calibrated torque wrench. With this known torque applied, knob 154′ will have moved rotationally relative to hub 158′ from its relaxed position. Once in this moved position, marks 1022 may be applied to knob 154′ directly adjacent to marks 1024 on hub 158′. When the predetermined torque is released, marks 1022 and 1024 will rotationally separate as knob 154′ returns to its relaxed position, as shown in FIG. 10. During use, the user merely needs to align marks 1022 with marks 1024 to obtain the precise torque desired. Marks 1022 can be applied during calibration by laser etching, mechanical engraving, painting, adhering a marker, melting a portion of knob 154′, or other such means. Alternatively, other methods of calibrating driver 132′ known to those skilled in the art may be used.

Tool shaft 162 may be configured to be rigid for simplicity and low cost. Alternatively, shaft 162 may be configured to be flexible so that it may access devices implanted in curved intramedullary spaces. This may be accomplished by constructing shaft 162 from a flexible material. However, it many circumstances, it desirable that tool shaft 162 only be flexible in a lateral bending direction, but as stiff as possible in tension, compression and torsion so that the tool is responsive during use. These goals may be accomplished by constructing shaft 162 from one or more layers of oppositely wound wire cable, or by using other composite assembly techniques or materials.

Driver tools 132 and 132′ described above provide ease of torque control for the user to limit the torque of device deployment. The tools increase resolution and reaction time for ceasing application of torque. These tools accurately control the tension on the implanted devices and the load on the bone when deployed, and increase patient safety. Because the tools are designed to be simple, they are inexpensive to manufacture. The tools may be designed and constructed to be sterilized for multiple uses, or they may be optimized for disposable, single-use.

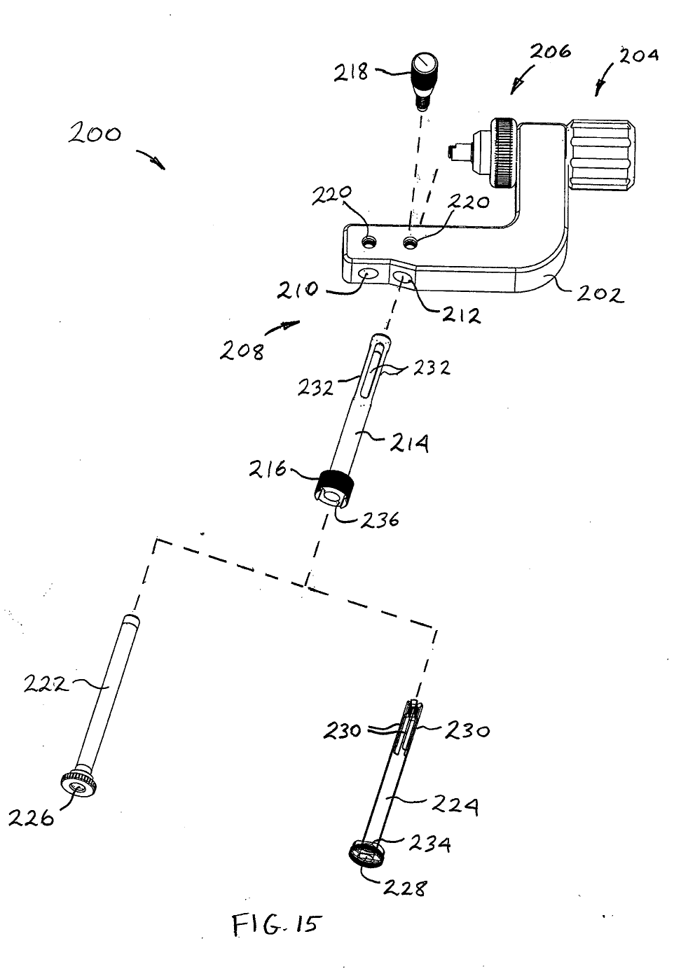

Referring to FIG. 15, a variation of the combination tool of FIG. 4 will now be described. Combination tool 200 includes a body 202, a device attachment portion 204, and an approximating driver 206. Since these components are similar in construction and operation to those on tool 138 described above, they will not be further described.

Combination tool 200 also includes a screw alignment portion 208, similar to that of tool 138. In this embodiment, tool 200 has a distal bore or aperture 210 and a proximal bore or aperture 212. Each of the apertures 210 and 212 is sized to receive an alignment sleeve 214. In some embodiments, each aperture 210 and 212 has its own alignment sleeve 214. In other embodiments, a single alignment sleeve 214 may be alternately placed in one of the two apertures 210 and 212 at any given time. Retaining sleeve(s) 214 may be provided with an enlarged head 216 on its proximal end to abut against tool body 202 when inserted through apertures 210 and 212. A retaining device such as a knurled thumb screw 218 may be used to thread through holes 220 in tool body 202 to secure alignment sleeve 214 within apertures 210 and 212.

In the exemplary embodiment of FIG. 15, a drill bushing 222 and a screw bushing 224 are provided, each to be alternately received within the central axial bore of alignment sleeve 214. Drill bushing 222 has an axial bore 226 for receiving a drill bit used to drill screw holes in the bone for securing the bone fixation device, as described above. Screw bushing 224 is configured with an axial bore 228 for receiving a bone screw and the shaft of a screw driver. Barbed fingers 230 longitudinally extending from the distal end of screw bushing 224 help retain the screw while it is being driven into the bone with the screw driver. Fingers 230 may flex radially outward when holding a screw, and may flex further outward when releasing the screw. Cutouts 232 may be provided through the distal end of alignment sleeve 214 to allow fingers 230 of screw bushing 224 to flex outward. In this embodiment, flats 234 are provided on the proximal head of screw bushing 224 to engage with keyway 236 on alignment sleeve head 216 to properly align screw bushing fingers 230 with alignment sleeve cutouts 232.

Referring to FIG. 16, an alternative embodiment of bone fixation device 300 is shown. Device 300 is similar in construction and operation to device 100 described above. Device 300 also includes a flexible-to-rigid body portion 302 having a generally helical slit 304 formed through the tube wall of that portion of the body. The helical slit 304 of this embodiment forms a T-shaped pattern such that the body portion adjacent to one side of slit 304 interlocks with the body portion on the directly opposite side of slit 304. The interlocking nature of this helical pattern allows device 300 to have only limited axial movement when subjected to axial tension loads. Axial tension loads may occur when a surgeon removes device 300 from the intramedullary space within a bone by pulling on the proximal end of device 300. In some embodiments, device 300 can withstand axial tension loads of up to 200 pounds or more. In some embodiments, device 300 has an outside diameter of about 5 mm and a length of about 100 mm.

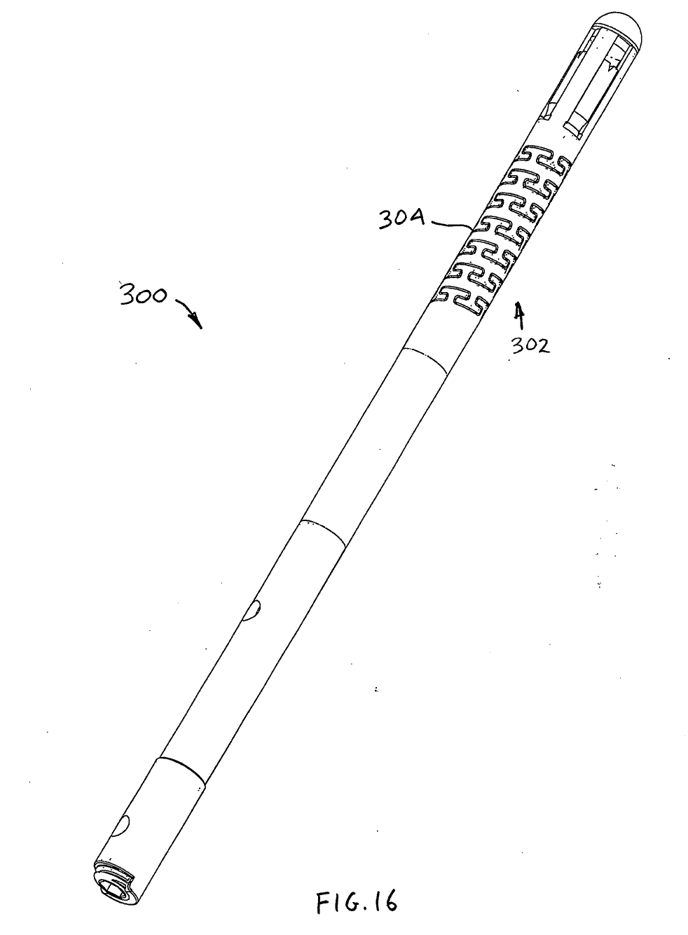

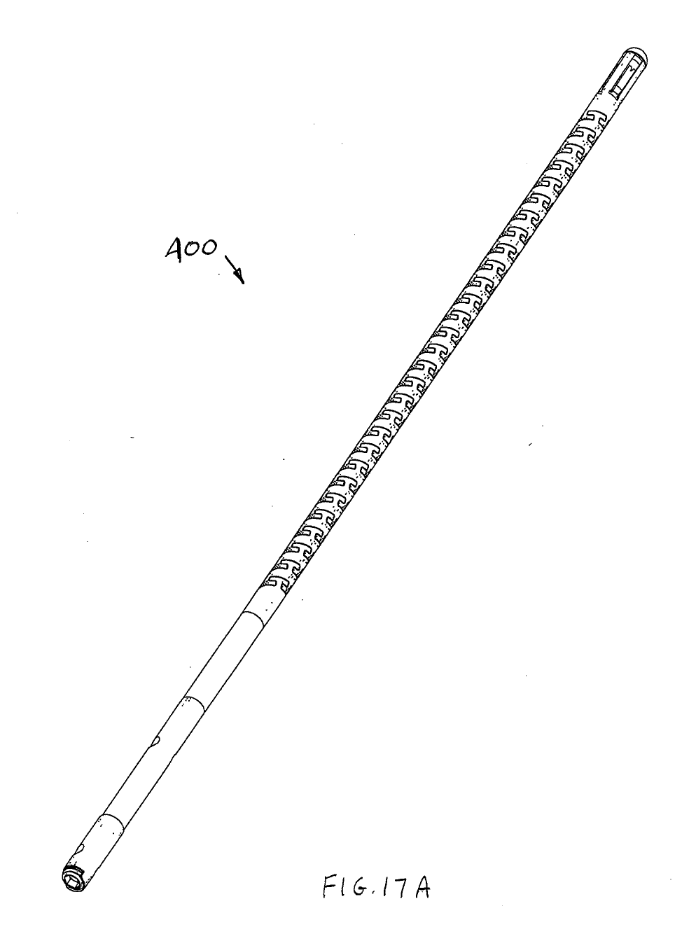

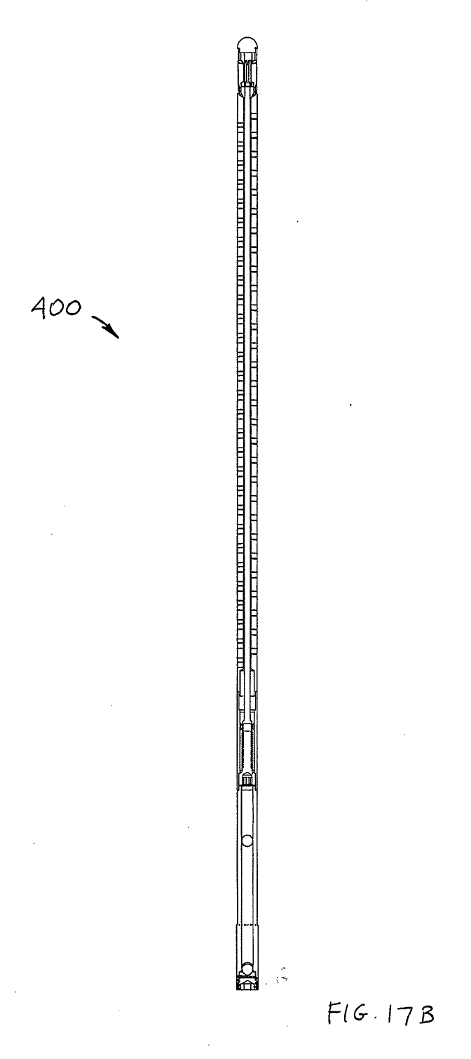

Referring to FIGS. 17A-17C, another embodiment of bone fixation device 400 is shown. Device 400 is similar to device 300 described above, but has a longer flexible-to-rigid body portion. Exemplary indications for devices 100 and 300 include fractures of the proximal ulna. In these situations, device 100 or 300 may be inserted into the intramedullary space of the proximal ulna through the olecranon, such as in the procedure described in detail below. Exemplary indications for device 400 include mid-shaft fractures of the ulna. In some embodiments, device 400 has an outside diameter of 4 mm and a length of 200 mm. In other embodiments, device 400 has an outside diameter of 5 mm and a length of 250 mm. Other sizes may be utilized to suit particular anatomies and injury or disease states. Other helical slit patterns on flexible-to-rigid body portions, different gripper locations, gripper types, and a different numbers of grippers (including no grippers) may also be utilized.

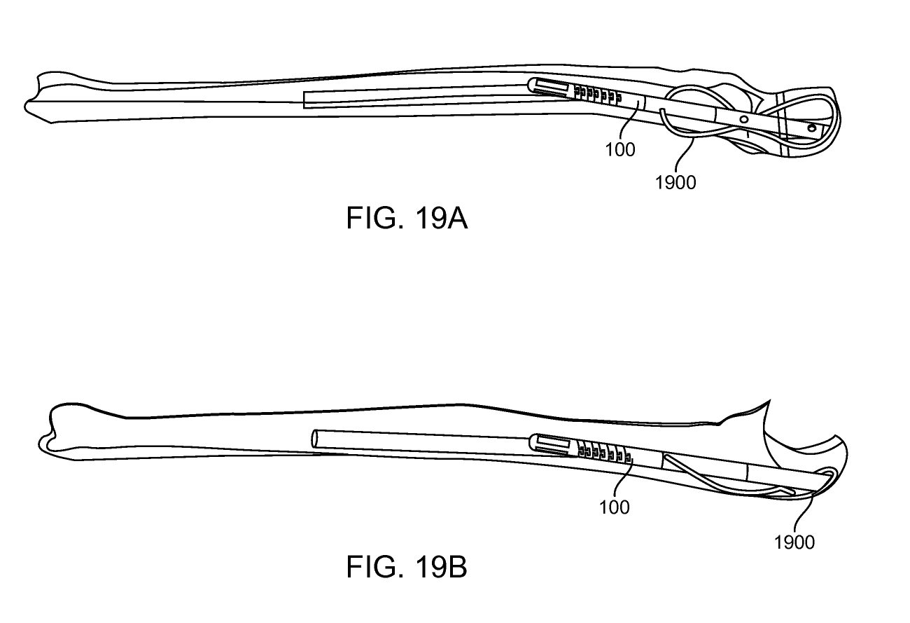

It is also envisioned in an alternate embodiment that a tension band in a figure-of-eight or other pattern be used to secure the entry point of the device to a position towards the hand. The tools described herein would provide for drilling one or more holes through the bone and/or the fixation device, and positioning either suture, wire, or other material so that a figure-of-eight or other pattern could be laced along the bone, through a distal hole (toward the hand) of the shaft of the ulna and around the orifice at the proximal (elbow) end of the device. An exemplary configuration of tension band 1900 is shown in combination with exemplary device 100 in FIGS. 19A and 19B.

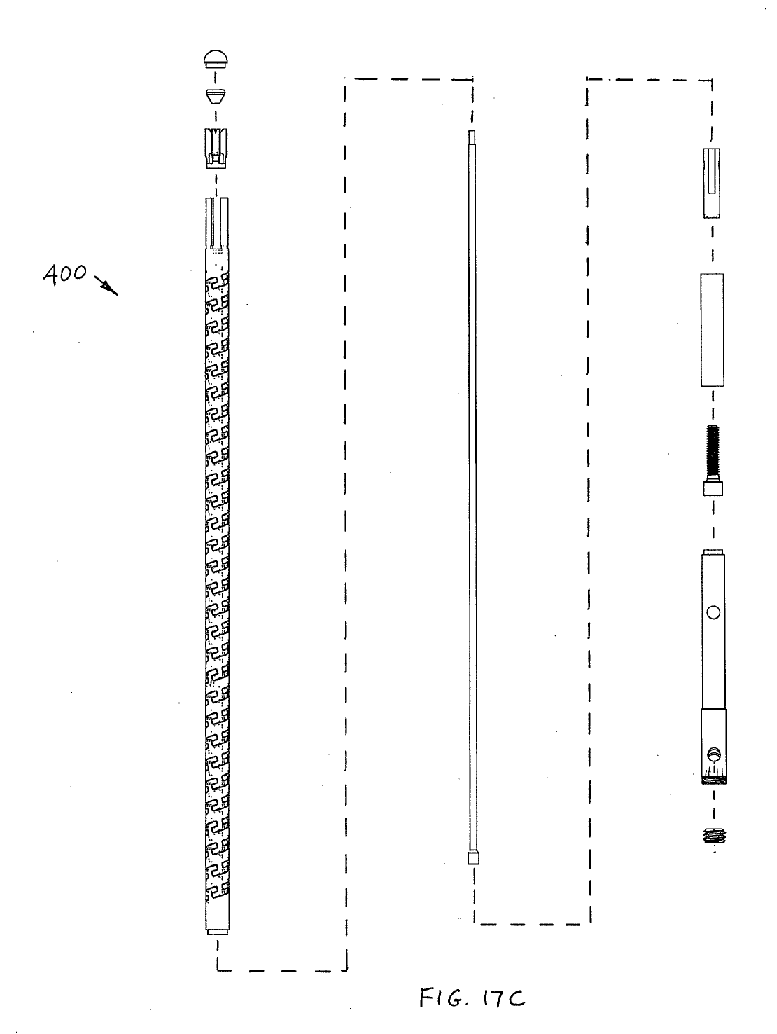

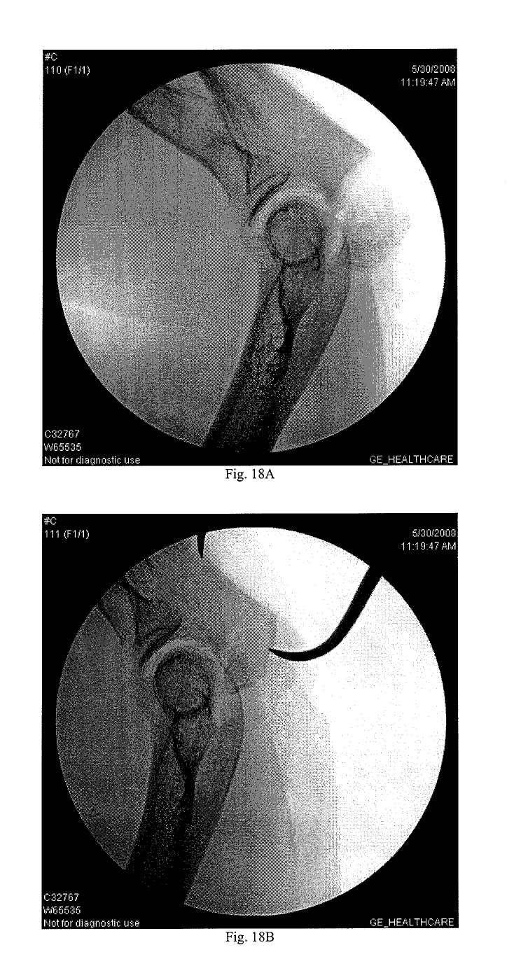

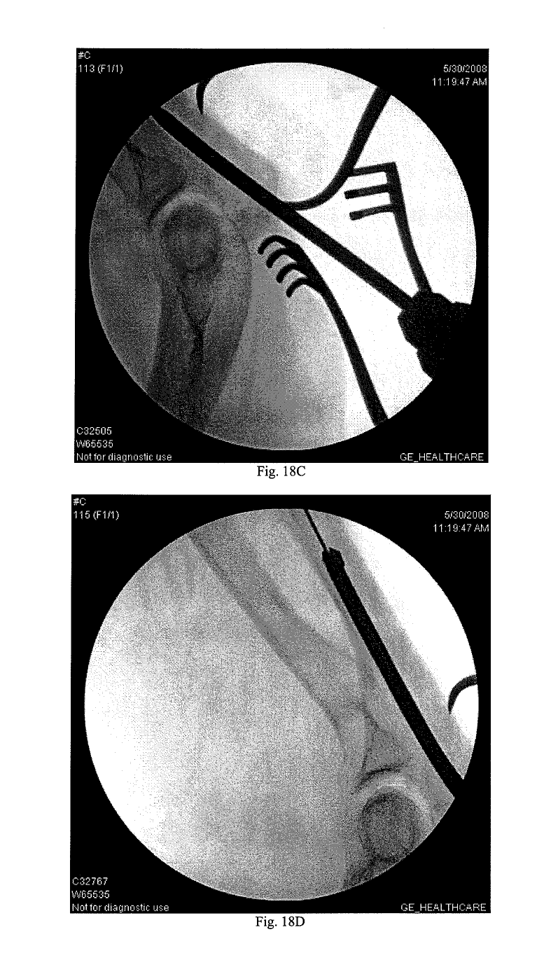

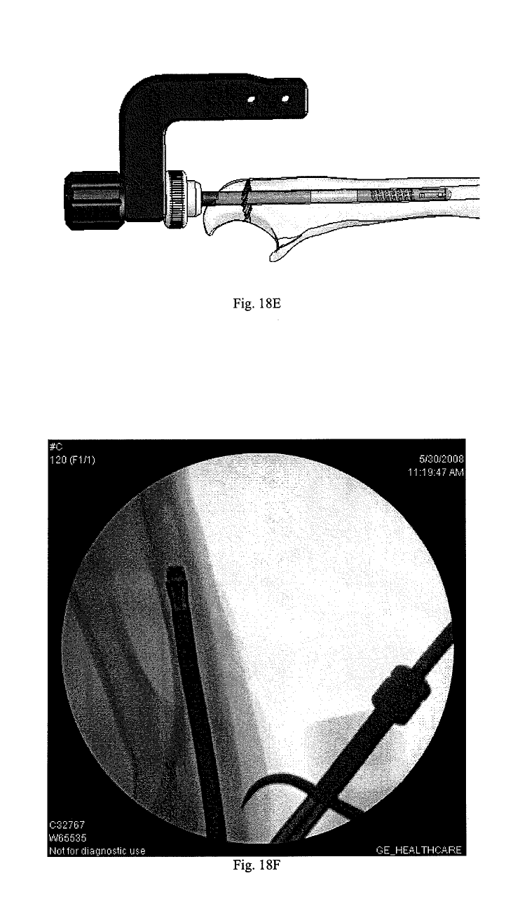

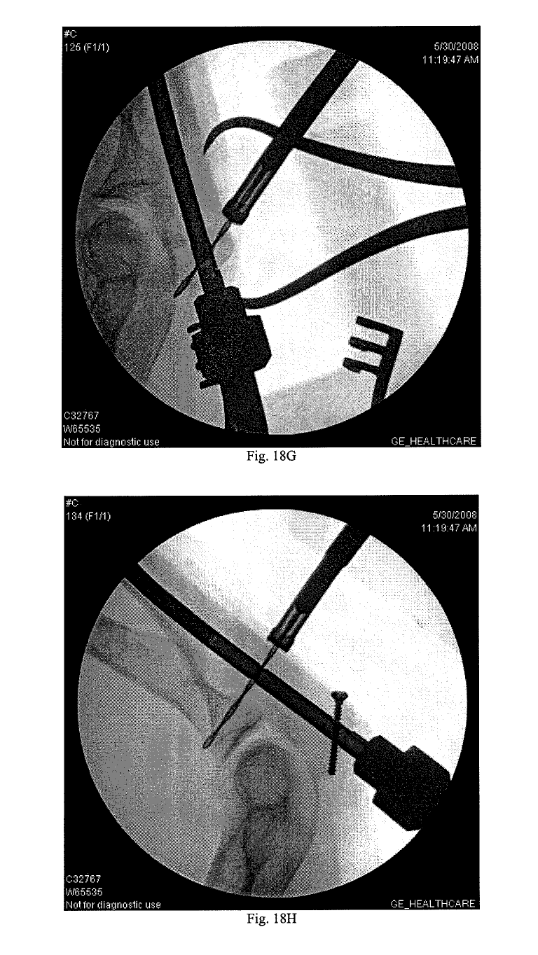

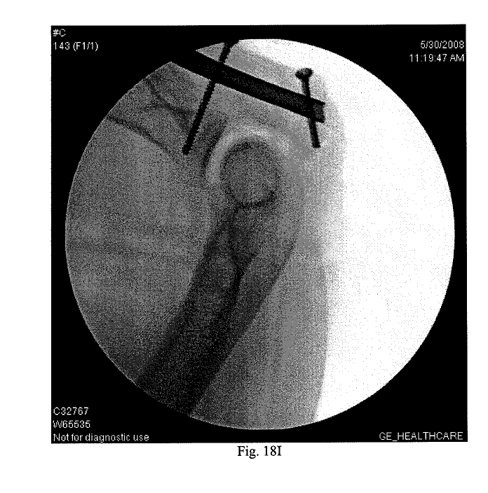

Referring to FIGS. 18A-18I, the following is an exemplary series of surgical steps that may be performed to implant the inventive devices disclosed in this application:
1. Ensure that the patient is a candidate for implantation of the intramedullary device and is properly prepared for surgical treatment.
2. Take an x-ray of the lateral elbow, such as shown in FIG. 18A. Ensure the fracture will span the hub between the proximal and distal screw holes.
3. Determine the length of the implant based on the point of varus angulation. The straight section of the implant should reside proximal to the point of varus angulation and the wavy body should extend into the curve in the canal.
4. Ensure that the implant and accessory tools are properly sterilized following parameters outlined in a product instruction manual.
5. Place the patient in either the surpine or the lateral position.
6. Using a scalpel, make a longitudinal incision 1-2 cm along the tip of the olecranon process.
7. Dissection is carried down sharply through the subcataneous tissues and the tricepts tendon. Care must be taken to avoid the ulnar nerve, which sits medial of the olecranon.
8. Reduce the fracture using standard surgical bone clamps or equivalent. Verify under fluoroscopy that the fracture is reduced and correct anatomical alignment is established, such as shown in FIG. 18B.
9. Establish the correct insertion site by perforating the cortex with a 2 mm drill. Start in the center of the olecranon process, directly in line with the proximal intramedullary canal of the ulna. Drill through the proximal fragment, across the fracture site and into the distal side of the fracture. Use fluoroscopy as required to ensure the correct path of the drill bit.
10. The entry will be enlarged using the appropriate sized drill bit for the implant. If cannulated drills are to be used, insert a guide wire through the proximal access hole, across the fracture and into the distal space.
11. Select the appropriate size drill for the implant that will be used. The drill should be 0.5 mm larger than the implant. For example, a 4.5 mm drill should be used with a 4 mm implant. With the appropriate drill, drill through the proximal access hole, across the fracture and into the distal side of the fracture, as shown in FIG. 18C.
12. If not already done, place a guide wire through the proximal access hole, across the fracture, and into the distal segment until the guide wire has followed the canal into the distal space.
13. Select the flexible reamer size appropriate for the implant. The correct reamer is about half a millimeter larger than the implant diameter. For example, a 5.5 mm reamer should be used with a 5.0 mm implant, and a 4.6 mm reamer should be used with a 4.0 mm implant. Maintain reduction of the fracture.
14. Attach the reamer to the driver and insert it over the guide wire, into the proximal access port and ream through the proximal fragment. Ream across the fracture site and into the distal space until the implant can be fully inserted, as shown in FIG. 18D.
15. Attach the outrigger to the implant. Maintain reduction and insert the implant into the bone.
16. Insert the device until the hub of the implant is just below the surface of the proximal cortex. Ensure the fracture spans the rigid section of the hub and that the location of the screw holes through, the hub does not interfere with the fracture.
17. Compress the fracture using the nut on the hub attachment tube, as shown in FIG. 18E.
18. Insert a torque driver into the hub. Rotate the torque driver clockwise to deploy the implant, as shown in FIG. 18F. Rotate the driver clockwise until the implant begins to feel snug, rotation may continue, as needed, until the lines on the shaft and handle of the driver are aligned. Do not rotate beyond this point.
19. Tug on the outrigger and ensure secure placement of the implant. Remove the torque driver,
20. Place the external drill guide sheath into the proximal location on the outrigger.
21. Place the drill guide into the external drill guide sheath. Make sure the sleeve is pushed forward and touching the bone—so the drill will be accurately positioned into the bone. Tighten the outrigger thumbscrew to prevent the external drill guide sheath from rotating.
22. Use the 2.0 mm drill to drill a hole through the bone and through the implant hub, as shown in FIG. 18G.
23. Use the depth gauge on the drill to measure the hole to the anterior cortex of the olecranon. Select the appropriate length 2.7 mm screw. Remove the drill and drill guide and insert the screw guide into the external sheath. Insert the screw through the guide and place the screw posterior to anterior with a phenolic screw driver handle and 2.5 mm hex bit.
24. Place the external drill guide sheath into the outrigger into the distal location on the outrigger.
25. Place the drill guide into the external drill guide sheath. Make sure the sleeve is pushed forward and touching the bone—so the drill will be accurately positioned into the bone. Tighten the outrigger thumbscrew to prevent the external drill guide sheath from rotating.
26. Use a 2.0 mm drill to drill a hole through the bone and through the implant hub, as shown in FIG. 18H.
27. Use the depth gauge on the drill to measure the hole to the anterior cortex of the olecranon. Select the appropriate length 2.7 mm screw. Remove the drill and drill guide and insert the screw guide into the external sheath. Insert the screw through the guide and place the screw posterior to anterior with the phenolic screw driver handle and 2.5 mm hex bit.
28. Remove the outrigger and take postoperative x-rays, as shown in FIG. 18I.

While exemplary embodiments of the present invention have been shown and described herein, it will be obvious to those skilled in the art that such embodiments are provided by way of example only. Numerous variations, changes, and substitutions will now occur to those skilled in the art without departing from the invention. It should be understood that various alternatives to the embodiments of the invention described herein may be employed in practicing the invention.

What is claimed is:

1. A surgical kit comprising:
a bone fixation device configured for implanting in an intramedullary space of a bone, the device comprising an elongated body having a first state in which at least a portion of the body is flexible and a second state in which the body is generally rigid, the device further comprising a mechanical actuator configured to change the device body between the first and second states; and
an actuator driver having an elongated shaft with a distal end configured to mate with the bone fixation device actuator, the driver being configured to indicate an amount of torque being applied from the driver to the actuator when the device is being changed from the first to the second state.

2. The surgical kit of claim 1 wherein the actuator driver is configured to limit the amount of torque that may be applied to the actuator.

3. The surgical kit of claim 1 wherein the bone fixation device further comprises at least one actuateable gripper disposed on the elongated body and movable by the actuator between a retracted state and an expanded state, wherein the actuator driver and the actuator cooperate to indicate an amount of torque being applied from the driver to the device body and the gripper.

4. The surgical kit of claim 1 wherein the shaft of the actuator driver is flexible.

5. The surgical kit of claim 1 wherein the actuator driver further comprises:
a hub connected to a proximal end of the shaft;
a knob rotatably coupled to the hub;
a spring element connected between the knob and the hub such that torque applied to the knob is transmitted through the spring to the hub, wherein the rotational position of the knob relative to the hub is related to the amount of torque being transmitted by the spring element; and
a marking feature on at least one of the knob and hub that indicates a rotational position of the knob relative to the hub.

6. A method of repairing a fracture of a bone, the method comprising:
inserting a bone fixation device into an intramedullary space of the bone to place at least a portion of an elongated body of the fixation device in a flexible state on one side of the fracture and at least a portion of a hub on another side of the fracture; and
using a torque indicating tool to operate a mechanical actuator on the bone fixation device after the inserting step, the actuator serving to change the elongated body from the flexible state to a rigid state.

7. The method of claim 6 wherein the operating of the actuator also serves to deploy at least one gripper of the fixation device to engage an inner surface of the intramedullary space to anchor the fixation device to the bone.

8. The method of claim 6 wherein the torque indicating tool comprises a flexible shaft.

9. The method of claim 6 further comprising inserting a screw through the bone and the hub.

10. A bone fixation device insertion tool comprising:
a tool body;
a sleeve attached to the body, the sleeve having a distal end configured to axially align with a proximal end of an elongated bone fixation device, the distal end of the sleeve having a keyed feature for engaging with a complementary mating feature on the proximal end of the fixation device to keep the fixation device in a predetermined rotational orientation relative to the sleeve;
a tube coaxially and rotably received within the sleeve, the tube having a distal end configured to engage with the proximal end of the fixation device to axially maintain the proximal end of the fixation device against the distal end of the sleeve, the insertion tool having a bore at least through a central axis of the tube for accessing a central axial bore of the fixation device; and
an screw alignment portion located on the tool body, the screw alignment portion having at least one aperture projecting onto a central longitudinal axis within the bone fixation device for guiding the placement of a screw into the device.

11. The insertion tool of claim 10 further comprising an approximating driver located on the sleeve, the driver configured to contact a bone and urge the bone in an axially distal direction from the sleeve.

12. The insertion tool of claim 11, wherein the approximating driver comprises an internally threaded knob that engages with an externally threaded portion of the sleeve, thereby causing the knob to move axially along the sleeve when the knob is rotated.

13. The insertion tool of claim 10 further comprising an alignment sleeve configured to be received within the aperture, wherein the screw alignment portion includes multiple apertures projecting onto the central longitudinal axis within the bone fixation device, each aperture being configured to alternately receive the alignment sleeve.

14. The insertion tool of claim 13 further comprising a drill bushing having an axial bore therethrough and configured to receive a drill bit in the axial bore, the drill bushing being further configured to be axially received in the alignment sleeve.

15. The insertion tool of claim 13 further comprising a screw bushing having an axial bore therethrough and configured to receive a screw in the axial bore, the screw bushing being further configured to be axially received in the alignment sleeve.

16. The insertion tool of claim 10 further comprising a torque driver having a shaft configured to be received in the bore of the tool, the torque driver shaft having a distal end configured to be received within the central axial bore of the bone fixation device and to engage with an internal actuator of the device, the driver being configured to indicate an amount of torque being applied from the driver to the actuator.
